(19) United States
(12) Patent Application Publication
(10) Pub. No.: US 2018/0311117 A1
(43) Pub. Date: Nov. 1, 2018

(54) FORMULATION COMPRISING ENCAPSULATED METAL OXIDE PARTICLES

(71) Applicant: Rohm and Haas Company, Collegeville, PA (US)

(72) Inventors: Fanwen Zeng, Audubon, PA (US); Pu Luo, King Of Prussia, PA (US); Xiaodong Lu, North Wales, PA (US); Junsi Gu, Malvern, PA (US)

(21) Appl. No.: 15/770,503

(22) PCT Filed: Nov. 8, 2016
(86) PCT No.: PCT/US2016/060881

§ 371 Date: Apr. 24, 2018

Related U.S. Application Data
(60) Provisional application No. 62/254,795, filed on Nov. 13, 2015.

(51) Int. Cl. A61K 8/02; A61K 8/27; A61K 8/29; A61K 8/81; A61K 8/92; A61Q 17/04

(52) U.S. Cl. CPC A61K 8/0241; A61K 8/27; A61K 8/29; A61K 8/8117; A61K 8/8147; A61K 8/8152; A61K 2800/624; A61K 8/92; A61Q 17/04; A61K 2800/412; A61K 2800/413; A61K 2800/43; A61K 8/8158

(57) ABSTRACT

An aqueous personal care composition comprising 0.5 to 10 wt % polymer encapsulated particles of titanium dioxide or zinc oxide; said polymer encapsulated particles comprising: (i) particles of titanium dioxide or zinc oxide having an average particle diameter from 10 to 200 nm; (ii) 0.25 to 10 wt % water-soluble sulfur acid-functional first polymer, based on weight of said particles of titanium dioxide or zinc oxide; and (iii) from 10% to 200%, by weight second polymer, based on weight of said particles of titanium dioxide or zinc oxide, wherein the second polymer at least partially encapsulates said particle of titanium dioxide or zinc oxide.

### BACKGROUND OF THE INVENTION

This invention relates to a formulation comprising metal oxide particles encapsulated in polymer.

Inorganic sunscreens are gaining popularity in personal care formulations for their capacity for providing broad spectrum UV protection and their hypoallergenic profile. Titanium dioxide and zinc oxide are the only inorganic sunscreen active ingredients approved by the FDA and most other regulatory agencies. U.S. Pat. No. 4,421,660 discloses encapsulation of inorganic particles, such as titanium dioxide or zinc oxide, by in-situ emulsion polymerization; the particles can be formulated into cosmetically or pharmaceutically acceptable formulations.

However, it remains technically challenging to develop inorganic sunscreen products, especially aqueous formulations, with high efficacy and consistency due to their tendency to flocculation and agglomeration.

### SUMMARY OF THE INVENTION

The present invention is directed to an aqueous personal care composition comprising 0.5 to 10 wt % polymer encapsulated particles of titanium dioxide or zinc oxide; said polymer encapsulated particles comprising: (i) particles of titanium dioxide or zinc oxide having an average particle diameter from 10 to 200 nm; (ii) 0.25 to 10 wt % water-soluble sulfur acid-functional first polymer, based on weight of said particles of titanium dioxide or zinc oxide; and (iii) from 10% to 200%, by weight second polymer, based on weight of said particles of titanium dioxide or zinc oxide, wherein the second polymer at least partially encapsulates said particle of titanium dioxide or zinc oxide.

### DETAILED DESCRIPTION OF THE INVENTION

As used herein, the following terms have the designated definitions, unless the context clearly indicates otherwise. An “aqueous” composition is one containing at least 50 wt % water. Unless otherwise specified, temperatures are in degrees centigrade (° C.), and references to percentages are percentages by weight (wt %). Operations are performed at room temperature (20-25° C.), unless otherwise specified. Percentages of components in the composition are on the basis of the entire composition.

Suitable shapes for the titanium dioxide or zinc oxide particles include spherical shapes, such as a regular sphere, an oblate sphere, a prolate sphere, and an irregular sphere; cubic shapes such as a regular cube and a rhombus; plate-like shapes including a flat plate, a concave plate, and a convex plate; and irregular shapes. Diameters of particles are defined as their maximum dimension. Preferably, the titanium dioxide or zinc oxide particles have average diameters of at least 15 nm; preferably no more than 150 nm, preferably no more than 120 nm, preferably no more than 100 nm, preferably no more than 60 nm. The average diameters of particles are typically provided by pigment particle suppliers and determined as weight averages. Preferably, the particles are titanium dioxide. Preferably, the particles are transparent. Particles may be uncoated or they may be surface treated, such as with alumina, silica, or organic materials such as esters. In some embodiments, the inorganic metal oxide particles are surface treated with alumina, alumina and jojobo esters, or alumina, jojobo esters, and silica. In some embodiments, the inorganic metal oxide particles are surface treated with alumina but are free of silica and jojobo esters.

Preferably, the titanium dioxide or zinc oxide particle encapsulated in polymer comprises at least 0.35 wt % water-soluble sulfur acid-functional first polymer, based on the weight of the titanium dioxide or zinc oxide particle, preferably at least 0.45 wt %, preferably at least 0.5 wt %; preferably no more than 8 wt %, preferably no more than 6 wt %, preferably no more than 5 wt %, preferably no more than 4 wt %, preferably no more than 3 wt %, preferably no more than 2 wt %. Typically the titanium dioxide or zinc oxide particle particles have been dispersed in an aqueous medium, with the water-soluble sulfur acid-functional first polymer. “Sulfur acid-functional polymer” herein includes any water-soluble polymer including at least three sulfur acid moieties. As used herein, the term “sulfur acid-functional monomer” is meant to include any monomer containing at least one free radical polymerizable vinyl group, and at least one sulfur acid moiety. As used herein, the term “sulfur acid moiety” is meant to include any of the following residues: —SO₂(OH), —OSO₂(OH), —OSO(OH), —SO(OH), and salts thereof. As used herein, the term “water-soluble sulfur acid-functional first polymer” means that the sulfur acid-functional first polymer is soluble in water at 25° C. at a pH no greater than 5 to an extent of at least 5 wt %.

The sulfur acid-functional first polymer can be any of a polymer with at least three sulfur acid moieties located randomly in the polymer backbone, a block copolymer with a single sulfur acid-including block and at least one block which does not have sulfur acids, or a comb-graft polymer with a backbone that includes sulfur acids and teeth which do not include sulfur acids. The block copolymers can have the sulfur acid-including block located terminal to the polymer, or within the interior of the polymer chain. In a preferred embodiment, the sulfur acid-functional polymer contains both sulfur acid and amine moieties. In this preferred embodiment, it is further preferred that the polymer have at least two amine and three sulfur acid groups, preferably at least three amine and five sulfur acid groups, preferably at least four amine and eight sulfur acid groups. The number of amine and sulfur acid groups may be the same or different. It is preferred that the molar ratio of amine to sulfur acid groups be from 10:1 to 1:10, preferably from 3:1 to 1:4, preferably from 1.5:1 to 1:3. The sulfur acid-functional polymer may be made as a solution polymer in water or a non-aqueous solvent, or as a bulk polymer. The sulfur acid-functional polymer may be made by any suitable polymerization process, such as addition polymerization of ethylenically unsaturated monomers such as acrylic, styrenic, or vinyl monomers. Polymers that contain both amine and sulfur acid groups may be made by copolymerizing at least one amine-functional monomer and at least one sulfur acid-functional monomer, or they may be made by including at least one monomer which is both amine-functional and sulfur acid-functional in the monomer mix. Examples of monomers that can be converted to amines after the polymerization is completed include isocyanate-functional monomers, which can be reacted with primary-tertiary or secondary-tertiary diamines, epoxy-functional monomers that can be reacted with amines, and halomethylbenzyl-functional monomers that can be reacted with amines. Examples of monomers that can be converted to sulfur acids after the polymerization is completed include isocyanate-functional monomers, which can be reacted with aminosulfates. Block copolymers that include a sulfur acid-functional polymer-including block may be made by any known process that is capable of producing such polymers. For example, block copolymers that include a sulfur acid-functional polymer-including block may be made by the living free radical polymerization of ethylenically unsaturated monomers wherein the monomer composition of one of the monomer feeds includes at least one sulfur acid-functional unsaturated monomer. As a further example, block copolymers that include a sulfur acid-functional polymer-including block may be made by the living free radical polymerization of ethylenically unsaturated monomers, including in the monomer mix functional monomers that can be converted to sulfur acid groups after the polymerization is completed. Comb-graft polymers that include a sulfur acid-functional polymer-including backbone may be made by any known process that is capable of producing such polymers. For example, comb-graft polymers that include a sulfur acid-functional polymer-including backbone may be made by the free radical polymerization of ethylenically unsaturated monomers wherein the monomer composition includes at least unsaturated macromer and at least one sulfur acid-functional unsaturated monomer. As a further example, comb-graft polymers that include a sulfur acid-functional polymer-including backbone may be made by the living free radical polymerization of ethylenically unsaturated monomers, including in the monomer mix functional monomers that can be converted to sulfur acid groups after the polymerization is completed. It is preferred that the sulfur acid-functional polymer be a linear random copolymer.

Suitable monomers for the first and second polymers include styrene (STY), butadiene, alpha-methyl styrene, vinyl toluene, vinyl naphthalene, ethylene, propylene, vinyl acetate, vinyl versatate, vinyl chloride, vinylidene chloride, acrylonitrile, methacrylonitrile, (meth)acrylamide, various C₁-C₂₀ alkyl esters of (meth)acrylic acid (e.g., methyl methacrylate (MMA), ethyl acrylate (EA), ethyl methacrylate (EMA), n-butyl acrylate (BA), 2-ethylhexyl acrylate (EHA), hydroxyethyl methacrylate (HEMA) and hydroxypropyl methacrylate (HPMA)); carboxylic acid containing monomers, such as acrylic acid (AA), methacrylic acid (MAA), itaconic acid, fumaric acid, and maleic acid. Examples of suitable sulfur acid-functional monomers include sulfoethyl (meth)acrylate, sulfopropyl (meth)acrylate, styrene sulfonic acid, vinyl sulfonic acid, and 2-(meth)acrylamido-2-methyl propanesulfonic acid, and salts thereof. Examples of suitable amine-functional monomers include dimethylamino ethyl(meth)acrylate (DMAEMA), dimethylamino propyl(meth)acrylamide, and t-butylamino ethyl(meth)acrylate. As used herein, the term “(meth)acrylate” refers to either acrylate or methacrylate and the term “(meth)acrylic” refers to either acrylic or methacrylic.

The sulfur acid-functional polymer random copolymer, sulfur acid including the block copolymer, or sulfur acid including backbone of the comb-graft polymer may have a weight average molecular weight of 1000 to 200,000, preferably from 1000 to 50,000, more preferably from 2000 to 15,000, and most preferably from 3000 to 10,000. When the sulfur acid-functional polymer is a block copolymer or a comb-graft polymer, the non-sulfur acid including block(s) or teeth, respectively, may have a weight average molecular weight of 750 to 200,000, more preferably from 1000 to 50,000, more preferably form 1500 to 25,000, and most preferably from 5000 to 15,000. The molecular weights are determined by GPC.

Preferably, the titanium dioxide or zinc oxide particle encapsulated in polymer comprises at least 30 wt % second polymer, based on the weight of the titanium dioxide or zinc oxide particle, preferably at least 50 wt %, preferably at least 70 wt %, preferably at least 80 wt %; preferably no more than 150 wt %, preferably no more than 130 wt %, preferably no more than 120 wt %. The second polymer is typically prepared by free radical emulsion polymerization of ethylenically unsaturated monomers in the presence of the pigment particle that has been dispersed in a medium. In a preferred embodiment, the second polymer is made of a monomer mixture containing at least one water-soluble monomer. Examples of suitable water soluble monomers are acid functional monomers including sulfur acid monomers as described above, acrylic acid, methacrylic acid, itaconic acid and the salts thereof. Other suitable water soluble monomers are acrylamide, diacetoneacrylamide, 2-hydroxyethyl methacrylate and 2-hydroxyethyl acrylate.

By “at least partially encapsulated” herein is meant that the second polymer is in contact with at least a part of the surface of the pigment particle. The degree of encapsulation of the pigment particle may be determined using an electron micrograph. Determination of the degree of encapsulation does not include any contribution of first polymer, surfactant or dispersant. By “X % encapsulated” herein is meant that X % of the surface area of the particle is in contact with the second polymer; preferably at least 50%, preferably at least 75%, preferably 100%. The thickness of the second polymer encapsulant layer may be up to 500 nm; for TiO₂ pigment, for example, preferred thickness of the second polymer encapsulant layer is typically between 20 nm and 150 nm, preferably from 40 nm to 100 nm.

The process for forming a particle encapsulated in polymer includes: (a) dispersing a titanium dioxide or zinc oxide particle in a medium with water-soluble sulfur acid-functional first polymer, preferably in the presence of a surfactant; and (b) performing an emulsion polymerization in the presence of the dispersed particle to provide a second polymer that at least partially encapsulates the particle.

In a preferred embodiment, the second polymer includes at least one sulfur acid-functional monomer. Examples of suitable sulfur acid-functional monomers include sulfoethyl acrylate, sulfoethyl methacrylate (SEM), sulfopropyl (meth)acrylate, styrene sulfonic acid, vinyl sulfonic acid, and 2-(meth)acrylamido-2-methyl propanesulfonic acid, and salts thereof. Preferably the sulfur acid-functional monomer is styrene sulfonic acid or its salt. The sulfur acid-functional monomer may be present at a level of from 0.1% to 20% by weight of the monomers used to make the second polymer containing the sulfur acid-functional monomer, preferably from 0.25% to 10%, more preferably from 0.25% to 5%, most preferably from 0.5% to 2%. If the second polymer contains more than one polymer phase, then the sulfur acid-functional monomer may be present in just some or in all of the polymer phases. If the second polymer contains more than one polymer phase, it is preferable that the sulfur acid-functional monomer is present in the first polymer stage to be polymerized.

Preferably, the second polymer contains only one phase. In one embodiment, the second polymer contains at least two phases, wherein one second polymer phase has a Tg at least 30° C., preferably at least 45° C., and at least one other second polymer phase has a Tg no greater than 12° C., preferably no greater than 0° C., preferably no greater than −5° C. In this embodiment, the one second polymer phase may be from 5 to 50 wt %, preferably from 10 to 40 wt %, preferably from 15 to 30 wt %, based on the pigment particle weight. Preferably, the total of the rest of the polymer phases of the second polymer is from 5 to 150 wt %, preferably from 10 to 125 wt %, preferably from 20 to 100 wt %, based on the pigment particle weight. Preferably, one phase comprises at least 50 wt % polymerized units of styrene sulfonic acid or its salts, preferably at least 75 wt %, preferably at least 80 wt %. Preferably, the first second polymer phase to be polymerized contains a multifunctional monomer. Suitable multifunctional monomers include, e.g., allyl (meth)acrylate, divinyl benzene, ethylene glycol di(meth)acrylate, propylene glycol di(meth)acrylate, trimethylolpropane tri(meth)acrylate, glycerol tri(meth)acrylate, and polyethylene glycol di(meth)acrylate. Preferably, the multifunctional monomer is present from 0.01 to 50 wt % based on total weight of the monomers which make up the first phase of the second polymer, preferably 0.1 to 10 wt %, preferably from 0.2 to 5 wt %, preferably from 0.2 to 2 wt %. In this embodiment, it is preferred that all of the phases of the second polymer have a Tg no greater than 5° C., preferably no greater than −5° C. In this embodiment, the first phase of second polymer may be from 5 to 50 wt %, preferably from 10 to 40 wt %, preferably from 15 to 30 wt %, based on the pigment particle weight. Preferably, the total of the rest of the polymer phases of the second polymer is from 5 to 150 wt %, preferably from 10 to 125 wt %, preferably from 20 to 100 wt %, based on the pigment particle weight.

Further details regarding encapsulation of inorganic particles may be found, e.g., in U.S. Pat. No. 8,283,404.

A personal care composition is a formulation containing at least one active ingredient, and intended for application to human skin. Suitable active ingredients include but are not limited to additional sunscreening actives (UV absorbers), moisturizing actives such as moisturizing oils, cleansing actives for personal care, detergent actives for personal care, thickeners, surfactants, film forming agents, mineral oil, silicones, colorants, vitamins, folic acid derivatives, exfoliating agents, deodorizing actives, fragrance actives, topical medicament actives for personal care, cosmetic agents for personal care, hair conditioners, facial care products, body washes, infrared (IR)-absorbing materials for personal care, acne medications and combinations thereof. Other additives may be included in the compositions of the invention such as, but not limited to, abrasives, absorbents, aesthetic components such as fragrances, pigments, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, and vitamins (e.g., Vitamin C) and derivatives thereof. Personal care compositions of the invention also include one or more dermatologically acceptable carriers. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. In some embodiments, the composition contains from about 99.99 to about 50 percent by weight of the dermatologically acceptable carrier, based on the total weight of the composition.

A particularly preferred personal care composition is a sunscreen. Preferably, the personal care composition comprises at least 1 wt % of the encapsulated titanium dioxide or zinc oxide particles of this invention (based on weight of encapsulated particle and total personal care composition weight), preferably at least 2 wt %, preferably at least 2.5 wt %, preferably at least 3 wt %; preferably no more than 8 wt %, preferably no more than 6 wt %. Preferably, the personal care composition comprises from 60 to 95 wt % water, preferably at least 70 wt %, preferably at least 80 wt %, preferably at least 85 wt %; preferably no more than 90 wt %. Preferably, the personal care composition comprises from 3 to 12 wt % of fatty alcohols, fatty acids, esters of fatty alcohols or fatty acids, or a combination thereof; preferably at least 5 wt %, preferably at least 6 wt %; preferably no more than 10 wt %. Fatty acids or alcohols, as the terms are used herein, are those having from twelve to twenty-two carbon atoms. Preferably, esters are glyceryl, polyethylene glycol or benzoate esters. Preferably, the personal care composition comprises from 0.1 to 3 wt % of a mono-fatty acid ester (preferably a C16-20 fatty acid) of a polyethylene glycol having from 30 to 50 polymerized units of ethylene oxide (preferably 35 to 45 units); preferably from 0.3 to 2.5 wt %, preferably from 0.5 to 2 wt %, preferably from 0.7 to 1.5 wt %. Preferably, the personal care composition comprises from 0.1 to 5 wt % of a polysiloxane, preferably at least 0.5 wt %, preferably at least 1 wt %; preferably no more than 4 wt %, preferably no more than 3 wt %. A preferred polysiloxane is dimethicone.

Suitable additional sunscreen actives include, for example, para aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, benzophenones, benzylidenes, salicylates, or other known UV filters, including diethanolamine methoxycinnamate, digalloy trioleate, ethyl dihydroxypropyl PABA, glyceryl aminobenzoate, and lawsone with dihydroxy acetone and red petrolatum.

Skin care compositions are generally administered topically by applying or spreading the compositions onto the skin. A person of ordinary skill in the art can readily determine the frequency with which the compositions should be applied. The frequency may depend, for example, on the level of exposure to UV light that an individual is likely to encounter in a given day and/or the sensitivity of the individual to UV light. By way of non-limiting example, administration on a frequency of at least once per day may be desirable.

### EXAMPLES

### Abbreviations

SDS=Sodium dodecylbenzene sulfonate (23%)
SSS=Sodium styrene sulfonate
TREM™ LF-40=Sodium dodecyl allyl sulfosuccinate (40%)
t-BHP=t-Butyl hydroperoxide
AIBN=azoisobutyronitrile
SSF=Sodium sulfoxylate formaldehyde
IAA=Isoascorbic acid
EDTA=Ethylene diamine tetra acetic acid sodium salt (VERSENE™)
nDDM=n-dodecylmercaptan
AMPS™=2-acrylamido-2-methylpropane-sulfonic acid
DI water=Deionized water

### Example 1. Preparation of Water-Soluble Sulfur Acid-Functional First Polymer

A monomer solution was made as 353.8 g of AMPS, 70.8 g of DMAEMA, 159.2 g of BA, 283.1 g of MMA, 19.5 g of nDDM, 354.9 g of ethanol, and 121.7 g of water. A 3 L flask equipped with a magnetic stirrer, N₂-inlet, reflux condenser, heating mantel, addition funnel, and thermocouple was charged with 104.4 g of water, and 222.7 g of ethanol. The flask was purged with N₂, and heated to 71° C. At 71 C, to the reaction flask was added 8.85 g VAZO™52 in 79.3 g ethanol, followed by monomer solution addition at a rate of 23 g/min for 60 min. After the addition of the monomer solution was completed the reaction mixture was held at 70° C. for 0.5 hours. The temperature was then raised to 77° C., and 8.85 g VAZO™52 in 79.3 g ethanol was added. The reaction mixture was held at 77° C. for 1 hr, then the flask was cooled to room temperature and the solvent was stripped off. The dried polymer was dissolved in sufficient water and NH₃ to make a 21.0% solution at pH 4.0.

### Example 2. Formation of TiO2 Dispersion

A steel grind pot was charged with 43.03 g of water, 416.50 g of polymer 1, 11.0 g of surfactant Rhodacal DS-4, and 3.94 g of defoamer (Foamstar A-34). Place the grind pot on a benchtop high speed dispersator in the fume hood and turn on the lowest speed. Begin adding 318 g of TiO₂ powder (Kobo TTO-S-4; average particle size 15 nm) adjusting the rpm as needed to get a good vortex. Once all the TiO₂ powder is added, scrape the pot and blade. Turn the speed up to achieve a good vortex and let grind for 25 min. The final TiO2 dispersion has approximately 40.1% of active TiO₂ and 11% of dry polymer 1.

### Example 3. Formation of Polymer-Encapsulated TiO₂ Particles

Monomer Emulsion (ME) was prepared by mixing water (40 g), Polystep A-16-22 anionic surfactant (5.16 g), butyl acrylate (BA, 28.28 g), methacrylic acid (MAA, 0.4 g), and methyl methacrylate (MMA, 170 g).

To a four-neck 2-L round bottom flask equipped with a mechanical paddle stirrer, a thermocouple, nitrogen inlet, and reflux condenser was added TiO₂ polymer composite slurry from example 2 (489 g) and water (30 g). The mixture was heated to 50° C. under N2; to the flask was sequentially added a premixed aqueous solution of sodium styrene sulfonate (1.66 g in 40 g water), and a mixture of an aqueous solution of iron sulfate heptahydrate (4.67 g, 0.15% iron), and an aqueous solution ethylene diamine tetraacetic acid (EDTA, 0.2 g, 1%). Cofeed catalyst (2.2 g t-butyl hydrogen peroxide in 60 g water) and cofeed activator (1.3 g isoascorbic acid in 60 g water) were fed to the flask at a rate of 0.54 g/min. After 2 minutes, ME was fed to the reactor at a rate of 2.7 g/min and the flask temperature was allowed to exotherm to 65° C. After completion of ME addition, the monomer emulsion vessel was rinsed with 5 g deionized water, which was added to the flask. The cofeed catalyst and activator addition was continued until completion. After completion of all feeds, the flask was cooled to room temperature. When the flask temperature reached 40° C., an aqueous solution of 29% aqueous ammonium hydroxide (5.8 g) and water (2 g) was added, followed by a solution of Rocima™ BT-2S (2.3 g) and water (6 g). After the flask was cooled to room temperature, the contents were filtered to remove any gel. The filtered dispersion was found to have a solids content of 40% with a pH of 9.

### Example 4: Sunscreen Formulation and Procedure

TABLE 1
The Sunscreen formulation

| | | 4A | 4B | 4C | 4D | 4E |
|---|---|---|---|---|---|---|
| Trade Name | INCI Name | w/w % | w/w % | w/w % | w/w % | w/w % |
| Phase I | | | | | | |
| DI Water | Water | 87.05 | 82.05 | 62.05 | 67.05 | 72.05 |
| Keltrol CGT | Xanthan Gum | 0.50 | 0.50 | 0.50 | 0.50 | 0.50 |
| Disodium EDTA | Disodium EDTA | 0.05 | 0.05 | 0.05 | 0.05 | 0.05 |
| Propylene Glycol | Propylene Glycol | 1.00 | 1.00 | 1.00 | 1.00 | 1.00 |
| Phase II | | | | | | |
| Ritox 52 (Rita) | PEG 40 Stearate | 1.00 | 1.00 | 1.00 | 1.00 | 1.00 |
| Rita GMS (Rita) | Glyceryl Stearate | 1.00 | 1.00 | 1.00 | 1.00 | 1.00 |
| Procol CS-20-D | Cetearyl Alcohol, Cetereth-20 | 3.00 | 3.00 | 3.00 | 3.00 | 3.00 |
| (Protameen) | | | | | | |
| Jeenchem TN | C₁₂₋₁₅ Alkyl Benzoate | 3.50 | 3.50 | 3.50 | 3.50 | 3.50 |
| (Jeen) | | | | | | |
| DC 200 Fluid, | Dimethicone | 2.00 | 2.00 | 2.00 | 2.00 | 2.00 |
| 100 cst | | | | | | |
| Phase III | | | | | | |
| Example 3 (20% | Polymer encapsulated TiO₂ | | | 25.00 | 20.00 | 15.00 |
| TiO₂): This | Dispersion | | | | | |
| invention | | | | | | |
| TTO-S-4 (Kobo); | Titanium Dioxide Stearic | | 5.00 | | | |
| comparative | Acid, Aluminum Hydroxide | | | | | |
| example | (un-encapsulated) | | | | | |
| Phase IV | | | | | | |
| Liquapar PE | Phenoxyethanol, | 0.90 | 0.90 | 0.90 | 0.90 | 0.90 |
| (Ashland) | Isopropylparaben, | | | | | |
| | Isobutylparaben, | | | | | |
| | Butylparaben | | | | | |
| Total | | 100.00 | 100.00 | 100.00 | 100.00 | 100.00 |

### Procedure:

In the main vessel, combine Phase I ingredients, mix until all dissolves and hydrates, heat to 75° C.; in a separate vessel, combine phase II ingredients, heat to 75° C. while mixing until all melts; add Phase II into Phase I with agitation, mixing until a uniformed emulsion is reached; at 75° C. add Phase III ingredients into the batch with fast agitation; after addition of Phase III, homogenize the batch at 4000 rpm for 3 minutes; continue mixing batch at moderate speed while cooling; when temperature below 40° C., add Phase IV into the batch, mix well while cooling to the ambient temperature. Example 4A and 4B are used as comparatives where 4A did not contain TiO₂, while 4B contained un-encapsulated TiO₂.

### Example 5: Evaluation of Sunscreen Formulation: In-Vitro SPF Test Procedure and Results

The SPF value of the sunscreen formulations were measured using an in-vitro technique substantially according to the following protocol in compliance with the COLIPA 2007 method:

First, the percent of solids of each formulation was measured using OHAUS MB45 solids analyzer. Second, the weight of a roughened PMMA substrate (purchased from SCHONBERG GmbH & Co. KG, Hamburg/Germany,) was measured. The sample to be tested was then spread on the substrate using a RDS #7 draw down bar to achieve a uniform layer. The layer was allowed to dry for about 20 minutes, and the weight of the substrate plus dry uniform layer is determined. The UV absorption between 290 nm and 400 nm of dry uniform layer was measured using a LABSPHERE UV-2000S Spectrometer at 9 points on the layer. Using the weight of the dry film and the solids content of the layer, the weight and the density of the original wet layer immediately after draw down can be calculated. The in-vitro SPF characteristic is described in the COLIPA 2007 guidelines as the ratio below (Labsphere “UV-2000S Ultraviolet Transmittance Analyzer” manual):

$$In \text{-} vitro SPF value = \frac{{\int }_{290 nm}^{400 nm} E ( λ ) S ( λ ) \partial λ}{{\int }_{290 nm}^{400 nm} E ( λ ) S ( λ ) {10}^{( - A ( λ ) )} \partial λ}$$

Where E(λ)=spectral irradiance of the Standard Sun Spectrum; S(λ)=erythemal action spectrum at wavelength λ; and A(λ)=corrected spectral absorbance at wavelength λ (a correction factor is calculated to extrapolate the data to establish what the absorbance would be at a wet layer density of 2.0 mg/cm2, using the original wet layer immediately after ∂ deposition).

All the formulations were let to be settled on bench for one week before in-vitro SPF measurement. Each formulation was measured 3 times according to the protocol described above. The results of in-vitro SPF measurement of the formulations, including Critical Wavelength, are shown in below:

TABLE 2
In-vitro SPF results:

| Formulation | SPF #1 | SPF #2 | SPF #3 | Average |
|---|---|---|---|---|
| 4A Base | 1.31 | 1.29 | 1.2 | 1.27 |
| 4B 5% TTO-S-4 | 4.73 | 5.33 | 4.3 | 4.79 |
| 4C 5% example 3 | 22.48 | 16.49 | 15.4 | 18.12 |
| 4D 4% example 3 | 11.71 | 9.88 | 13.65 | 11.75 |
| 4E 3% example 3 | 7.59 | 7.77 | 9.2 | 8.19 |

TABLE 3
Critical Wavelength (nm)

| Formulation | CW #1 | CW #2 | CW #3 | Average |
|---|---|---|---|---|
| 4A Base | 388.78 | 389 | 388.89 | 388.89 |
| 4B 5% TTO-S-4 | 378.89 | 379.78 | 379.89 | 379.52 |
| 4C 5% example 3 | 375.67 | 377.11 | 374.89 | 375.89 |
| 4D 4% example 3 | 375.11 | 375.33 | 375.67 | 375.37 |
| 4E 3% example 3 | 376.44 | 376.11 | 375 | 375.85 |

### Example 6: Evaluation of Sunscreen Formulation: Hiding Test Procedure and Results

The hiding test is to evaluate the aesthetic feature of a skin care products. in most sun care products, consumers prefer high transparency, which is difficult to achieve for inorganic sunscreen formulations. Hiding test result is represented by contrast ratio. The higher the contrast ratio, the higher the opacity of the product is, and the more whitening of a product will be perceived after being applied on skin.

Hiding test procedure is described as below:

Using a 3MIL bird wet film applicator (BYK), make a drawdown film of each of the sunscreen formulation to be tested on a black/white drawdown card (Leneta Form 2A); let the drawdown film dry completely in a controlled room (75F/50% RH); using BYK Spectro-Guide 45/0, measure the L value of each of the drawdown film on both black and white side. The contrast ratio is calculated by the following equation:

Contrast Ratio(CR)=L(Black)/L(White)

TABLE 4
The results of hiding test are shown below

| | | | 4B 5% | | 4C 5% | | 4C 4% | | 4C 3% | | | |
|---|---|---|---|---|---|---|---|---|---|---|---|---|
| | 4A Base | | TTO-S-4 | | example 3 | | example 3 | | example 3 | | Blank | |
| L | B | W | B | W | B | W | B | W | B | W | B | W |
| L | 11.0 | 92.4 | 49.4 | 93.0 | 49.2 | 92.9 | 46.2 | 92.6 | 40.7 | 92.5 | 6.0 | 92.1 |
| #1 | | | | | | | | | | | | |
| L | 11.2 | 92.4 | 48.6 | 93.1 | 48.7 | 92.9 | 46.0 | 92.7 | 39.9 | 92.4 | 6.0 | 92.1 |
| #2 | | | | | | | | | | | | |
| avg | 11.1 | 92.4 | 49.0 | 93.1 | 49.0 | 92.9 | 46.1 | 92.7 | 40.3 | 92.5 | 6.0 | 92.1 |

| | | | | | | |
|---|---|---|---|---|---|---|
| CR | 0.12 | 0.53 | 0.53 | 0.50 | 0.44 | 006 |

The hiding test results indicate that for a given SPF, sunscreen formulation with encapsulated TiO₂ will provide higher transparency than un-encapsulated TiO2.

### Example 7. Comparison of Sunscreen Formulation Containing Un-Encapsulated TiO₂ (Example 4B) and Encapsulated TiO₂ (Example 4C) by Both Cryogenic Transmission Electron Microscopy (Cryo-TEM) and Scanning Electron Microscopy (SEM)

### Procedure

Successful encapsulation of TiO₂ particles by polymer coating was demonstrated by cryogenic transmission electron microscopy (cryo-TEM). The micrographs were acquired in a Hitachi H-7000T transmission electron microscope equipped with a tungsten filament at 125 kV. The TEM samples were prepared by a cryo dilute emulsion preparation method. In brief, two drops of the slurry sample were diluted in 15 mL of DI water and nebulized onto a Cu grid at −75° C. The sample holder was then inserted into the microscope for imaging while being maintained at the same temperature.

The dispersion quality of TiO₂ particles in the dry film of the sunscreen formulation was determined by scanning electron microscopy (SEM). The SEM samples were prepared by drop casting small droplets (˜1 mm in diameter) of formulated sunscreens onto an aluminum Q-panel. The samples were let dry in ambient condition overnight and were coated with carbon using a Denton Bench Top Turbo III coater. The imaging was carried out in a FEI Nova NanoSEM 630 electron microscope equipped with a zirconiated tungsten field emission electron source operated at 5 kV. The micrographs were acquired using a low voltage high contrast backscatter electron detector (vCD) at 1024×884 resolution with an e-beam of spot size 4. The TiO₂ cluster size was measured based on the grayscale line profile of the scanning electron micrographs. All image analyses were performed in ImageJ (version 1.49u).

### Results

From cryo-TEM, TiO₂ treated with polymer encapsulation in this invention showed consistent polymer encapsulation. The treatment did not break the TiO₂ into the finest primary particles, but instead it isolated the primary particles into ˜100 nm clusters with multiple primary particles encapsulated in one cluster. In the scanning electron micrographs, TiO₂ clusters were visualized as bright spots/regions within the darker matrix of sunscreen film. The aggregation of TiO₂ cluster size was significantly reduced in the encapsulated sample. The image analysis showed a decrease in average mean TiO₂ cluster size from 235 nm to 166 nm as a result of the encapsulation technology. The cluster size reduction will lower the scattering efficiency of the sunscreen film towards visible light, thus minimizing the undesirable skin whitening caused by the sunscreen.

Scanning electron micrographs of sunscreen formulations using the unencapsulated TiO₂ and the polymer-encapsulated TiO₂ displayed the following cluster sizes.

TABLE 5
TiO₂ cluster size in sunscreen film measured
from the scanning electron micrographs.

| | Unencapsulated | Encapsulated |
|---|---|---|
| Sample | TiO₂ | TiO₂ |
| TiO₂ Cluster size (Mean)/nm | 235 | 166 |
| TiO₂ Cluster size (Medium)/nm | 160 | 102 |

1. An aqueous personal care composition comprising 0.5 to 10 wt % polymer encapsulated particles of titanium dioxide or zinc oxide; said polymer encapsulated particles comprising: (i) particles of titanium dioxide or zinc oxide having an average particle diameter from 10 to 200 nm; (ii) from 0.25 to 10 wt % water-soluble sulfur acid-functional first polymer, based on weight of said particles of titanium dioxide or zinc oxide; and (iii) from 10% to 200%, by weight second polymer, based on weight of said particles of titanium dioxide or zinc oxide, wherein the second polymer at least partially encapsulates said particle of titanium dioxide or zinc oxide.

2. The formulation of claim 1 in which each of the first and second polymers comprises polymerized units of styrene, alpha-methyl styrene, vinyl toluene, vinyl naphthalene, acrylonitrile, methacrylonitrile, (meth)acrylamide, C₁-C₂₀ alkyl esters of (meth)acrylic acid, (meth)acrylic acid, itaconic acid, fumaric acid, maleic acid, sulfoethyl (meth)acrylate, sulfopropyl (meth)acrylate, styrene sulfonic acid, vinyl sulfonic acid, 2-(meth)acrylamido-2-methyl propanesulfonic acid, amine-containing (meth)acrylate monomer, salts thereof and combinations thereof.

3. The formulation of claim 2 comprising from 1 to 8 wt % of said polymer encapsulated particles of titanium dioxide or zinc oxide.

4. The formulation of claim 3 comprising from 80 to 95 wt % water and from 3 to 12 wt % of fatty alcohols, fatty acids, esters of fatty alcohols or fatty acids, or a combination thereof.

5. The formulation of claim 4 in which said encapsulated particles comprise from 0.5 to 5 wt % water-soluble sulfur acid-functional first polymer.

6. The formulation of claim 5 in which said encapsulated particles comprise from 50% to 150%, by weight second polymer.

7. The formulation of claim 6 in which said particles of titanium dioxide or zinc oxide have an average particle diameter from 10 to 150 nm.

8. The formulation of claim 7 comprising from 2 to 8 wt % of said polymer encapsulated particles of titanium dioxide or zinc oxide.

9. The formulation of claim 8 in which the particles are titanium dioxide.
